(19) United States
(12) Patent Application Publication
(10) Pub. No.: US 2021/0282660 A1
(43) Pub. Date: Sep. 16, 2021

(54) Device for Locating a Medical Implant

(71) Applicant: Neuroloop GmbH, Freiburg (DE)

(72) Inventors: Dennis PLACHTA, Vörstetten (DE); Tim BORETIUS, Freiburg (DE); Fabian KIMMIG, Teningen (DE)

(21) Appl. No.: 17/258,064

(22) PCT Filed: Jun. 26, 2019
(86) PCT No.: PCT/EP2019/067006

§ 371 Date: Jan. 5, 2021

(30) Foreign Application Priority Data
Jul. 6, 2018 (DE) 10 2018 211 185.5

(51) Int. Cl. A61B 5/06; A61M 5/142

(52) U.S. Cl. CPC A61B 5/061; A61M 5/14276

(57) ABSTRACT

A device is described for the transcutaneous location of an intracorporeally, subcutaneously located medical implant, which has an implant surface on which at least one electrical coil is arranged. The coil has at least one coil winding enclosing a region of the implant surface, together with an extracorporeally operable unit, to which at least one electrical coil is fitted, which is adapted in shape and size to the electrical coil arranged on the implant, and which is connected to an electrical control unit and to a signalling means, which generates a perceptible signal, at least in the case of maximum inductive coupling between the two electrical coils.

### CROSS REFERENCE TO RELATED APPLICATIONS

Reference is made to PCT/EP2019/067006 filed Jun. 26, 2019, designating the United States, which claims priority to German Application No. 10 2018 211 185.5 filed Jul. 6, 2018, which are incorporated herein by reference in their entirety

### BACKGROUND OF THE INVENTION

### Field of the Invention

The invention relates to a device for the transcutaneous location of an intracorporeally, subcutaneously located medical implant, which has an implant surface, on which at least a first electrical coil is arranged, which has at least one coil winding enclosing a region of the implant surface. For the purpose of locating the coil winding, an extracorporeally operable unit is used, on which is fitted at least one electrical coil, which is connected to an electrical control unit and to a signalling device, and with induction detection a maximum possible coil overlap between the intra and extracorporeal coil can be detected.

### Description of the Prior Art

EP 1 052 935 B1 provides a system and a method for the location of an implementable drug delivery device, which is implanted directly under the skin. The drug delivery device comprises, amongst other items, a drug reservoir, for which the reservoir wall provides a septum, which in the implanted state is oriented towards the skin surface for purposes of multiple refilling of the reservoir. The filling of such an implanted drug reservoir requires a precise knowledge of the location and position of the subcutaneously located plenum, so that a doctor can hit the underlying plenum without further patient stress, and with only a single puncture through the skin with the aid of an injection needle, in order to be able to undertake the filling process with the aid of an injection syringe device.

In order to find the exact position of the intracorporeally located septum, an electrical coil surrounding the septum is provided on the implant side, which is connected to an electrical energy source housed within the drug delivery device as well as an electronic controller, and can be operated as a transmitting antenna. With the aid of an extracorporeally operable implant-locating antenna arrangement, which has three air-coil antennas evenly distributed along a circular line, the position of the transmitting antenna on the implant side and thus the septum enclosed by the latter can be located. For this purpose, the extracorporeal implant-locating antenna array must be moved over the surface of the patient's skin until the three air-coil antennas of the locating antenna array, evenly distributed along the virtual circular line, are concentrically aligned with the implant-side transmitting antenna.

In addition to the locating antenna array, optical signalling means, for example in the form of illuminable arrow symbols, serve as navigation aids for the doctor. The illumination of the arrows is based on electrical energy components inductively coupled into each of the three air coils. If the electrical energy received by all three air coils is the same, the implant-locating antenna arrangement is located concentrically above the transmitting antenna fitted to the implant. In the center of the implant-locating antenna array a passage guide is fitted to aid the penetration of an injection needle, so that the doctor can locally puncture the underlying septum for the purpose of filling the drug reservoir.

US published patent application 2008/028127 A1 describes a device for the transcutaneous location of an intracorporeally positioned implant, which provides an implant-locating antenna arrangement that can be operated extracorporeally and that can be moved over the surface of the skin to locate an intracorporeally positioned coil, in a similar manner to the locating system described above in EP 1 052 935 B1.

US published patent application 2017/0100056 A1 discloses a communication system between an intracorporeally-positioned coil assembly and an extracorporeally-arranged movable coil array, which is able to transmit signal data as well as energy between a single extracorporeally-positioned coil and an intracorporeally-positioned coil. For this purpose, the intracorporeal coil has a larger coil diameter than the individual extracorporeal coils provided in an array arrangement.

### SUMMARY OF THE INVENTION

The invention is based on the development of a device for the transcutaneous location of an intracorporeally, subcutaneously located medical implant, which has an implant surface on which is arranged at least a first electrical coil, which has at least one coil winding enclosing a region of the implant surface, and with an extracorporeally operable unit, to which is fitted at least one electrical coil, which is connected to an electrical control unit and to a signalling device, such that the time expenditure to be managed by a doctor, as well as the level of concentration required for the manual centring of the extracorporeally operable unit relative to the electrical coil fitted to the implant is minimized. At the same time, it is important to ensure that a subsequent penetration, through the skin and the inner region of the electrical coil fitted to the implant, can be carried out reliably and accurately with the aid of an injection needle. In particular, it is important to relieve the doctor of the burden of carrying out the above-cited measure on the one hand, and to protect the patient from possible penetration failures on the other.

The device in accordance with the invention provides transcutaneous location of an intracorporeally, subcutaneously located medical implant, for electrical coils including a first coil fitted to the implant and further coils with the further coils having diameters similar to the first coil fitted to the implant so as to be equal to or less than a coil diameter of the first coil assigned to the electrical coil fitted to the implant. In addition, the electrical further coils fitted to the extracorporeally operable unit are connected to the electrical control unit and to the signalling device, or in each case to a signalling device, which, at least in the case of maximum inductive coupling between at least one of the electrical coils and the electrical coil fitted to the implant, for generating a perceptible signal identifying at least the electrical coil with maximum inductive coupling.

The device according to the invention avoids the sometimes time-consuming search process for the doctor to find the exact position of the electrical coil fitted to the implant, in which the doctor has to carry out a highly precise centering process by controlled relative movements of the known extracorporeally operable unit while observing navigation information, which is preferably optical, that is perceptible to the doctor. The invention instead provides for a surface substrate which is a mat or film as an extracorporeally operable unit, of a skin-compatible and flexible material, which is preferably transparent to light, in which the electrical coils with a highest possible spatial packing density are arranged.

The electrical coils are preferably arranged on or within the mat of the surface substrate along a first plane of arrangement, indirectly, or preferably directly, adjacent to one another, so that the proportion of surface area within the first plane of arrangement that is not covered by at least one electrical coil is as small as possible, and is preferably minimized.

The surface shape and size of the mat or film surface substrate, in which the electrical coils, at least in some regions, are at least in some regions are distributed evenly and are arranged to cover the surface as homogeneously as possible, are preferably selected such that by merely applying or placing the surface substrate on the skin surface in a region of a very probable position of the subcutaneously located electrical coil fitted to the implant, at least one of the electrical coils fitted within the surface substrate comes to be in a position above the electrical coil located on the implant.

For the purpose of an exact location of the subcutaneously located electrical coil fitted to the implant, the control unit, which is also fitted directly, or indirectly, to the mat-form surface substrate, compares the electrical energies coupled from induction in all the electrical coils fitted to the surface substrate, and determines that electrical coil, or that group, composed of a plurality of electrical coils, in which the maximum electrical energy can be measured, in each case by way of a maximum inductive coupling as a result of maximum overlap with the coil on the implant.

With a signalling device, preferably in the form of an illuminant, the electrical coil, or group of coils, that couples with the implanted electrical coil with maximum inductance, is immediately indicated visually. In a preferred embodiment, each electrical coil is provided with an illuminant within the mat surface substrate, so that with the illumination of one of the illuminants a perceptible coil identification is possible. The illuminant preferably takes the form of an LED, or a fluorescent or phosphorescent material, which is directly fitted to each electrical coil, or is incorporated in the coil material.

The illuminant fitted to each of the plurality of electrical coils preferably emits electromagnetic waves with wavelengths in the visible spectral range. For this reason, the skin-compatible material from which the mat-form surface substrate is made, and which surrounds the electrical coils at least partially in a matrix, is made of a light-transparent material, preferably a polyamide, or a silicone-based plastic material. The surface substrate, which flexibly adapts to the shape of the body surface, also allows a largely unhindered insertion of an injection needle, so that after the identification of at least one electrical coil with maximum inductive coupling with the electrical coil fitted to the implant, a doctor can insert an injection needle as centrally as possible, without hindrance, within a circular area that is surrounded by the electrical coil identified with the aid of the respective illuminant, through both the surface substrate and the septum, which in projection is located directly under the skin surface, for the purpose of filling.

In order to ensure that the coil identified with maximum inductive coupling is, in projection onto the skin surface, always is arranged above the electrical coil fitted to the implant, the coil diameter of each of the electrical coils fitted to the surface substrate is selected to be, as a maximum, equal to, preferably less than, the coil diameter of the electrical coil fitted to the implant. All electrical coils arranged on the surface substrate have the same coil diameter.

A further preferred example of embodiment of the extracorporeally operable unit provides at least a second set of electrical coils within the mat surface substrate, which are arranged next to one another along a second plane of arrangement, parallel to the first plane of arrangement, directly or indirectly adjacent, or partially overlapping in pairs such that in an orthogonal projection onto the first and second planes of arrangement the electrical coils of the two planes of arrangement in each case partially overlap one another. This ensures that in the case of inductive coupling between one of the plurality of electrical coils and the electrical coil fitted to the implant, the electrical coil arranged on the surface substrate, which by virtue of its maximum inductive coupling is perceptible in appearance, preferably visually, compared to all the other electrical coils, is positioned exactly above or within the electrical coil fitted to the implant.

### BRIEF DESCRIPTION OF THE DRAWINGS

In what follows the invention is described, without any limitation of the invention, with the aid of examples of embodiments, with reference to the drawings. Here:

FIG. 1 shows a representation of a mat surface substrate with electrical coils, and an electrical coil fitted to the implant, and

FIG. 2 shows an example of embodiment of a mat surface substrate with electrical coils fitted in a distributed manner in two planes of arrangement.

### PATHS TO THE EMBODIMENT OF THE INVENTION, INDUSTRIAL Applicability

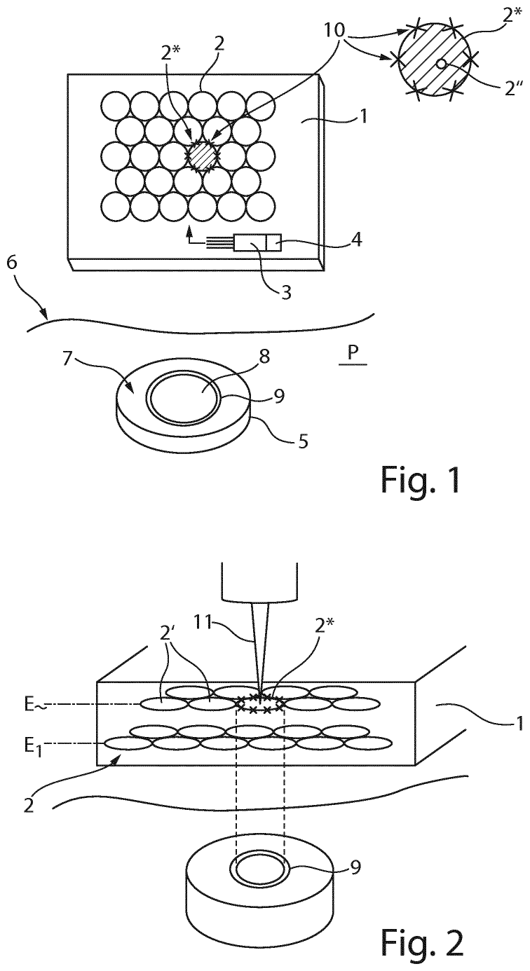

FIG. 1 illustrates a mat surface substrate 1, which is made of a flexible, skin-compatible material, preferably a silicone-based plastic, in which electrical coils 2 of the same shape and the same size are integrated. All the electrical coils 2 are electrically connected to a control unit 3, which is directly or indirectly connected to an electrical energy source, preferably being in the form of a battery or an accumulator 4. The electrical coils 2 can be arranged directly adjacent to one another along a plane of an arrangement within, or on, the surface substrate 1, as shown in FIG. 1. In this case, intermediate regions are formed between the coils, which are not enclosed by the electrical coils. In order to minimize or eliminate such intermediate regions as completely as possible, it is alternatively possible to arrange the electrical coils 2 with a mutual overlap along a plane of arrangement.

The mat-form surface substrate 1 is positioned on the skin surface 6 of a patient P for the purpose of locating an implant 5. The medical implant 5, in particular, takes the form of a drug pump arrangement, which has an implant surface 7 in which at least one self-sealing port 8 is introduced in the form of a septum, which seals a reservoir volume located within the medical implant in a fluid-tight manner. In the implanted state, the implant surface 7 is oriented essentially parallel to the skin surface 6, so that the septum 8 is arranged close to the skin surface 6. In addition, the septum 8 is surrounded by an electrical coil 9, which can be activated by way of a control unit contained within the medical implant 5 (not shown further) as well as an electrical energy source in the form of a transmitting antenna.

For the purpose of locating the septum 8, the mat surface substrate 1 illustrated in FIG. 1 is applied to the skin surface 6 of the patient P in the region of the medical implant 5, covering the surface and following the contours. The surface dimensions of the surface substrate 1, together with the surface arrangement and extent of the coils 2, are chosen such that a doctor, even when uncertain as to the exact position of the implant, can attach the surface substrate 1 onto the surface of the patient's skin such that at least one coil 2 overlaps geometrically with the coil 9 fitted to the implant, without having to correct the position of the surface substrate subsequently.

The electrical coils 2 arranged on the mat surface substrate 1 each serve as receiving coils, which are able to receive and absorb electrical energy in the course of an inductive coupling, which, with the aid of the control unit 3, can be detected, measured, and compared, between the individual electrical coils. From the electrical coils 2, coil in the substrate is determined, which has maximum inductive coupling with the electrical coil 9 fitted to the implant, and thus has the maximum electrical energy. Alternatively, the electrical coils arranged on the mat surface substrate can be operated actively, that is to say, as transmitting coils, and the at least one electrical coil fitted to the implant can be operated passively. In this way, the electrical energy source serving the implant is not under load during the location process. In this case also, the electrical coil fitted to the mat surface substrate, which has a maximum inductive coupling with the implant-side electrical coil, is visually indicated.

Each of the electrical coils 2 integrated into the mat-form surface substrate 1 is separately fitted with an illuminant 10, as for example, an LED. For the indication and visual perception of that electrical coil 2* with the maximum inductive coupling among all the electrical coils present, the illuminant 10 of the same electrical coil 2* is activated by means of the control unit 3, so that a doctor can quickly and clearly detect, by means of the illuminated electrical coil 2*, the region of the surface substrate through which the injection needle must be inserted, in order to puncture the septum 8 located immediately below it. FIG. 1 shows the illuminated coil 2*, with the indicated illuminant 10 separately enlarged.

The injection needle should preferably be positioned centrally within the circular area 2″, which is radially bounded by the respectively illuminated electrical coil 2.

FIG. 2 shows an example of a mat surface substrate 1, in which electrical coils 2 are arranged along a first plane of arrangement E1, comparable to the example shown in FIG. 1, that is to say, the electrical coils 2 arranged in the first plane of arrangement E1 are of the same size and are arranged in an array directly adjacent to one another. Furthermore, further electrical coils 2′ are arranged in a second plane of arrangement E2, which is oriented parallel to the first plane of arrangement E1, wherein the coils 2′ within the second plane of arrangement E2 are each offset by one coil having half-diameter with respect to the coils 2 within the first plane of arrangement E1. In a projection orthogonal to both planes of arrangement E1, E2, the electrical coils 2, 2′ from the two different planes of arrangement E1, E2 accordingly overlap. This increases the reliability with which at least one coil can be orthogonally positioned exactly above and within the electrical coil 9 fitted to the implant, so that a doctor, when, with the aid of an injection needle 11, pushing through the inner area of the electrical coil 2*, illuminated with the aid of a suitable illuminant, punctures exactly the septum 9 of the implant located intracorporeally directly underneath.

The device in accordance with the invention for determining the transcutaneous location of an intracorporeally subcutaneously prescribed medical implant avoids any searching movements and allows an immediate display and identification of the electrical coils which are located above the intracorporeally located septum by placing the mat surface substrate, which is provided with the electrical coils, on the skin surface just the once. The surface substrate has a surface that is preferably conditioned to prevent slipping on the skin surface, for example, is suitable for the formation of an adherent surface joint that is free of adhesive material.

### LIST OF REFERENCE SYMBOLS

1 Mat-form surface substrate
2 Electrical coils
2′ Electrical coils of the second plane of arrangement
2* Illuminated electrical coil
3 Control unit
4 Electrical energy source
5 Medical implant
6 Skin surface
7 Implant surface
8 Septum
9 Electrical coil arranged on the implant
10 Illuminant
11 Injection needle
E1, E2 First and second planes of arrangement

1.-10. (canceled)

11. A device for determining a transcutaneous location of an intracorporeally subcutaneously located medical implant, comprising an implant surface on which at least a first electrical coil is arranged, at least one coil winding enclosing a region of the implant surface, together with an extracorporeally operable unit, on which at least one electrical coil is fitted, which is connected to an electrical control unit and to a signalling device;
the extracorporeally operable unit including additional electrical coils each with coil diameters which are dimensioned to a coil diameter of the first coil and to be equal to or less than a coil diameter of the electrical coil fitted to the implant; and wherein
the additional electrical coils are connected to the electrical control unit and to the signalling device, or to the signalling device, and wherein at least in a circumstance of maximum inductive coupling between at least one of the electrical coils on the extracorporeal operable unit and the at least one electrical coil fitted to the implant, a perceptible signal is generated identifying at least the electrical coil with a maximum inductive coupling.

12. A device according to claim 11, wherein:
the extracorporeally operable unit has a surface substrate on or in which the further electrical coils is located.

13. A device according to claim 12, wherein:
at least the electrical coils fitted to the extracorporeally operable unit are located relative to one another along a first plane, or are partially overlapping in pairs.

14. A device according to claim 13, wherein:
at least a second set of the electrical coils fitted to the extracorporeally operable unit are located adjacent to one another along a second plane, parallel to a first plane of the arrangement, or are partially overlapping in pairs so that in an orthogonal projection onto the first and second planes of the electrical coils of the first and second planes which partially overlap one another.

15. A device according to claim 11, wherein:
the signalling means is an illuminant.

16. A device according to claim 15, wherein:
the illuminant is fitted to each electrical coil so that when one of the illuminants is illuminated, a perceptible identification of a single electrical coil is possible.

17. A device according to claim 15, wherein:
the illuminant is an LED, a fluorescent material, or a phosphorescent material.

18. A device according to claim 11, wherein:
the medical implant has at least one reservoir volume, which is bounded at least by at least one region of the implant surface enclosed by the coil winding, and the region comprises a self-sealing port for filling an injection device.

19. A device according to claim 18, wherein:
the region comprises a self-sealing septum.

20. A device according to claim 11, wherein:
the surface substrate includes a material transparent to light and has an adhesive surface for detachably adhesion to a surface of skin.
